(19) United States
(12) Patent Application Publication
(10) Pub. No.: US 2006/0177886 A1
(43) Pub. Date: Aug. 10, 2006

(54) Method for diagnosis of rheumatoid arthritis

(76) Inventors: Tadahiko Hazato, Asaka-shi (JP); Yukio Yamamoto, Nagareyama-shi (JP); Yoshiro Yamamura, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 11/209,700

(22) Filed: Aug. 24, 2005

(30) Foreign Application Priority Data
Feb. 5, 2005 (JP) 2005-052007

(51) Int. Cl. C12Q 1/37

(52) U.S. Cl. 435/23

(57) ABSTRACT

The present invention aims at providing a method for the diagnosis of RA. The present invention relates to a method for the diagnosis of rheumatoid arthritis, comprising measuring the dipeptidyl peptidase activity in a sample collected from a mammal.

### BACKGROUND OF THE INVENTION

1. Field of the Invention

The present invention relates to a method for the diagnosis of rheumatoid arthritis. More specifically, the present invention relates to a method for the diagnosis of rheumatoid arthritis by analyzing a dipeptidyl peptidase and spinorphin, which are expressed proteins in a sample collected from a subject.

2. Description of the Related Art

Rheumatoid arthritis (hereinafter abbreviated as RA) is a disease characterized by chronic multijoint synovial inflammation, and progressive destruction of cartilages and bone tissues. It is known that increase in the concentration of many protein-degrading enzymes (e.g., matrix metalloproteinase (MMP), cathepsin, peptidase and the like) decompose proteoglycan and collagen in the cartilage tissue (see Landewe R. and other seven authors, Arthritis Rheum, 2004, Vol. 50, pp. 1390-1399, Tchetverikov I. and other seven authors, An Rheum Dis, 2003, Vol. 62, pp. 1094-1099). Furthermore, it is known that interleukin-1β (IL-1β), tumor necrosis factor-α (TNF-α), various cytokines and enzyme inhibiting factors play important roles in the etiology of RA (see Cross A. and other three authors, Arthritis Rheum, 2004, Vol.50, pp.1430-1436, Raap T. and other five authors, J Rheumatol, 2000, Vol. 27, pp. 2558-2565). These proteins relate to knee and joint intricately, and make worse the condition from chronic inflammation to joint destruction. For the medication of RA patients, non-steroid antiinflammatory drugs (NSAIDs), disease-modifying antirheumatic drugs (DMARDs) and biological drugs (anti-TNF receptors and IL-1 antagonists) and the like are used (see Klimiuk P A. and other three authors, J Rheumatol, 2004, Vol. 31, pp. 238-242, Dougados M. and other six authors, J Rheumatol, 2003, Vol. 30, pp. 2572-2579, De A. and other seven authors, J Rheumatol, 2002, Vol. 29, pp. 46-51). However, despite of intensive treatments, conditions of many RA patients are not recovered, and the patients continue to feel pain and to receive bone destruction. The pain of RA is considered to be chronic and invasive, and is a signal that stimulates various nociceptive receptors in peripheral regions (knees and joints) and is transferred to the cerebrum via the spinal cord and perceived by the cerebrum.

As mentioned above, RA is a disease that can lead to serious condition. However, current diagnosis method of RA is based on the “ACR Revised Diagnosis Criteria” by the American College of Rheumatology (ACR). The criteria of the diagnosis are 1) morning stiffness (mainly in hands and fingers), lasting 1 hour or more; 2) swelling of 3 or more joint areas; 3) swelling in hand joints (wrist joints, metacarpophalangeal joints, proximal phalanx joints); 4) swelling of symmetric joints (the same joint areas of the left and right hands); 5) abnormal observation in an X-ray photograph of hands; 6) subcutaneous nodule and 7) positive response of RA in blood test. A patient shall be said to have RA if he/she has satisfied with 4 or more criteria. In the blood test, rheumatoid factor (RF) in serum is mainly measured. RF is a self-antibody against Fc portion of IgG that appears frequently in the serum of an RA patient. RF in a healthy person is a polyactive antibody that reacts with various proteins other than IgG, while a monoactive RF that reacts with only IgGFc exists in RA patients besides the polyactive RF. The reactivity of the monoactive RF to IgGFc is about 100-fold higher than that of polyactive RF. Accordingly, said RF is used in the measurement as an index for the determination of RA patients. However, there are RA patients who are suffering from serum reaction-negative RA in which RF is always not detected. In addition, it is known that about 2% of healthy persons can exhibit positive reaction.

Accordingly, a method for the determination of RA, comprising subjecting urine collected from an RA patient to liquid chromatography to detect the chromatogram peak of a component specific to RA (see JP-A-1995-72133), a method for the diagnosis of RA, comprising cleaving a sugar chain from a sugar protein, labeling the sugar chain and purifying the labeled sugar chain to prepare a sample for the analysis of the sugar chain, analyzing the prepared sample by high performance liquid chromatography using an ODS-silica column to give a composition of the sugar chain in the sample, and diagnosing RA based on the change in the composition of the sugar chain (see JP-A-1996-228795) and the like have been suggested.

However, these methods are insufficient because the methods show peaks higher than those observed in a healthy person, and preparation of samples for the analysis in the methods are complicated.

### SUMMARY OF THE INVENTION

The present invention aims at providing a method for the diagnosis of RA.

The pain of RA is considered to be chronic and invasive, and is a signal that stimulates various nociceptive receptors in peripheral regions (knees and joints) and is transferred to the cerebrum via the spinal cord and perceived by the cerebrum. A neuropeptide called as spinorphin (LVVYPWT; sequence listing: SEQ ID NO: 1) was isolated. Spinorphin is an endogenous peptide derived from bovine spinal cord, which plays a role in the activation of anti-inflammatory and anti-invasive properties. It is known that spinorphin suppresses bradykinin (BK)-induced nociceptive pain reaction in animal models, and that spinorphin suppresses various functions of polymorphonuclear neutrophils (PMNs), such as chemotaxis, O₂ generation, exocytosis and the like.

In order to elucidate the role of spinorphin in the control of inflammation and pain, the present inventors have done various studies with a focus on the change in activities of spinorphin and metabolic enzymes in the cerebrospinal fluid (CSF) from RA patients suffering from chronic pain and inflammatory condition, thereby to find that the amount of spinorphin, and the activation of the dipeptidyl peptidase in CSF changed in RA patients.

Furthermore, the present inventors have compared the activity of the dipeptidyl peptidase and the amount of spinorphin using synovial fluid collected from joints of RA patients and patients suffering from osteoarthritis (hereinafter abbreviated to as OA) in order to investigate the roles of the activity of the dipeptidyl peptidase and spinorphin in inflammation and nociceptive pain reaction mechanism. As a result, the inventors have found that the activity of the dipeptidyl peptidase increases and the amount of spinorphin decreases in an RA patient, and that a correlation exists between the increase of the activity of the dipeptidyl peptidase and the decrease of the amount of spinorphin, which resulted in the completion of the present invention.

Namely, the present invention relates to:
(1) a method for the diagnosis of rheumatoid arthritis, comprising measuring the activity of the dipeptidyl peptidase in a sample collected from a mammal;
(2) the method for the diagnosis according to the above-mentioned (1), wherein the sample is at least one member selected from joint synovial fluid, blood and cerebrospinal fluid;
(3) the method for the diagnosis according to the above-mentioned (1) or (2), further comprising measuring the amount of spinorphin in the sample collected from a mammal;
(4) the method for the diagnosis according to the above-mentioned (3), wherein rheumatoid arthritis disease is estimated at a spinorphin concentration of not more than 5 ng/mL in the measured sample;
(5) a method for measuring the activity of a dipeptidyl peptidase for the diagnosis according to the above-mentioned (1), comprising reacting a fluorescence substrate specific to a dipeptidyl peptidase and an aminopeptidase inhibitor with a sample in the presence or absence of a dipeptidyl peptidase inhibitor, and measuring the fluorescence intensity of the reaction solution after the reaction;
(6) the measurement method according to the above-mentioned (5), wherein the fluorescence substrate is a synthetic fluorescence substrate having an Arg-Arg group;
(7) the measurement method according to the above-mentioned (6), wherein the synthetic fluorescence substrate is Arg-Arg-4-methylcoumaryl-7-amide;
(8) the measurement method according to the above-mentioned (7), comprising measuring the fluorescence intensity of 7-amino-4-methylcoumarin produced by the reaction;
(9) the measurement method according to any one of the above-mentioned(5) to (8), wherein the aminopeptidase inhibitor is bestatin;
(10) the measurement method according to any one of the above-mentioned (5) to (9), wherein the dipeptidyl peptidase inhibitor is tynorphin;
(11) a kit for the diagnosis of rheumatoid arthritis, comprising a fluorescence substrate specific to a dipeptidyl peptidase, an aminopeptidase inhibitor and a dipeptidyl peptidase inhibitor;
(12) the kit according to the above-mentioned (11), wherein the fluorescence substrate is a synthetic fluorescence substrate having an Arg-Arg group; and
(13) The kit according to the above-mentioned (12), wherein the synthetic fluorescence substrate is Arg-Arg-4-methylcoumaryl-7-amide.

According to the present invention, the activity of the dipeptidyl peptidase in a sample, especially in the synovial fluid collected from the joint of RA patients is specifically higher than that of an OA patient. This means that the activity of the dipeptidyl peptidase in a sample can be used as a criterion for the diagnosis of RA. Accordingly, measurement of the activity of the dipeptidyl peptidase in a sample for the estimation and diagnosis of RA can be used as a novel method of the diagnosis of RA.

Furthermore, the amount of spinorphin is low, i.e., 5 ng/mL in a sample, especially in the synovial fluid collected from the joint of an RA patient. This means that the amount of spinorphin in the sample can be used as a criterion of the diagnosis of RA.

Accordingly, by measuring the amount of spinorphin besides the above-mentioned measurement of the activity of the dipeptidyl peptidase for the estimation and diagnosis of RA, the accuracy of the diagnosis of RA can be improved, and said method can be used as a novel method for the diagnosis of RA.

### BRIEF DESCRIPTION OF THE DRAWINGS

FIG. 1 shows the activity of the dipeptidyl peptidase in the synovial fluid collected from OA patients and RA patients.

FIG. 2 shows the amount of spinorphin in the synovial fluid collected from OA patients and RA patients.

### DETAILED DESCRIPTION OF THE PREFERRED EMBODIMENTS

Hereinafter, the method for the diagnosis of RA of the present invention is explained.

The sample that can be used for the present invention is not specifically limited so long as it is collected from a mammal. Examples of the sample may include samples from a living body such as cerebrospinal fluid, joint synovial fluid, blood, plasma, serum, saliva and urine, and various organs and tissues and the like collected from animals such as humans, rats, mice, dogs, cattle, cats, rabbits, guinea pigs and the like. Among them, joint synovial fluid, blood and cerebrospinal fluid are preferred.

The activity of the dipeptidyl peptidase in a sample can be measured by reacting the sample with a reaction solution comprising a fluorescence substrate specific to the dipeptidyl peptidase and an aminopeptidase inhibitor in the presence or absence of the dipeptidyl peptidase inhibitor, and measuring the fluorescence intensity of the reaction solution after the reaction.

Firstly, a reaction solution is prepared. There are prepared a reaction solution obtained by adding a fluorescence substrate and an aminopeptidase inhibitor to a buffer having a pH suitable for the activity of the dipeptidyl peptidase, preferably a pH of about 7 to 9, and a reaction solution further comprising a dipeptidyl peptidase inhibitor besides the above-mentioned buffer, fluorescence substrate and aminopeptidase inhibitor.

Examples of the buffer may include, for example, Tris hydrochloric acid buffer, phosphate buffer and the like.

Examples of the fluorescence substrate may include, for example, synthetic substrates having an Arg-Arg group such as Arg-Arg-MCA, Boc-Gly-Arg-Arg-MCA, Suc-Ala-Ala-Phe-AMC and the like. As used herein, MCA means 4-methylcoumaryl-7-amide, Boc means t-butoxycarbonyl group, Suc means succinyl group and AMC means 7-amino-4-methylcoumarin. Hereinafter MCA, Boc and AMC have the same meanings as mentioned above.

The amount of the fluorescence substrate to be added is preferably an amount sufficient to react with the dipeptidyl peptidase in the sample, and is generally preferably about 0.2 to 5 μM.

Preferable examples of the aminopeptidase inhibitor may include bestatin, leuhistin and the like. The aminopeptidase inhibitor is added so that the reaction of aminopeptidase with the above-mentioned substrate can be inhibited and that the substrate specificity of the dipeptidyl peptidase to the substrate can be increased. Thus, the enzyme activity of the aminopeptidase in the sample can be inactivated. Therefore, the amount of the aminopeptidase inhibitor is preferably an amount sufficient for suppressing the aminopeptidase activity in the sample, and is generally preferably about 50 to 250 μg/mL.

It is preferable that the synthetic fluorescence substrate is preincubated with the aminopeptidase inhibitor at about 30 to 40° C., preferably about 37° C., for about 1 to 30 minutes.

It is preferable that the dipeptidyl peptidase inhibitor is an inhibitor that specifically inhibits the dipeptidyl peptidase to be used, and example thereof may include tynorphin and the like. The amount of the dipeptidyl peptidase inhibitor to be used is preferably an amount sufficient to inhibit the activity of the dipeptidyl peptidase in the sample, and is generally preferably about 50 to 200 μg/mL.

It is preferable that the reaction of the sample and the above-mentioned reaction solution is incubated at about 20 to 40° C., preferably at about 37° C., for usually about 10 to 60 minutes, preferably about 20 to 40 minutes. By such incubation, for example, in the case where Arg-Arg-MCA is used as a fluorescence substrate, the fluorescence substrate liberates 7-amino-4-methylcoumarin (AMC) in a reaction solution by an enzyme (dipeptidyl peptidase). This reaction can be represented by the equation: Arg-Arg-MCA→Arg-Arg+AMC.

Secondly, it is preferable that the reaction is stopped by the addition of a reaction stopping solution (e.g., acetic acid, sodium acetate and the like) to the above-mentioned reaction solution.

After the reaction is stopped, the fluorescence intensity of the liberated reaction product is measured. The wavelength for the measurement of the fluorescence intensity differs depending on the reaction product. For example, in the case where the reaction product is the above-mentioned AMC, it can be measured at the excitation wavelength of 360 nm and the fluorescence wavelength of 440 nm.

The activity of the dipeptidyl peptidase in the sample can be explained by the following equation:
Activity=(Activity of substrate degradation in the absence of a dipeptidyl peptidase inhibitor)−(Activity of substrate degradation in the presence of a dipeptidyl peptidase inhibitor).

Such activity can be expressed in terms of the activity per 1 mg of the protein relative to the total amount of the protein in the sample, i.e., the unit of pmol/incubation period (min)/mg protein.

The total amount of the protein in the sample can be measured by known methods for measuring proteins, e.g., by the Bradford method (Pierce Biotechnology, Inc., Rockford, USA) and the like.

The above-mentioned activity of the substrate degradation can be converted to a value per 1 mg of the protein in the sample by depicting a standard curve based on the relationship between the concentration of AMC and the value of fluorescence, and calculating the value from the standard curve.

Although the activity of the dipeptidyl peptidase obtained by the measurement based on the above-mentioned method varies depending on the conditions of the measurement (reaction temperature, reaction period and the like) and the like, RA can be diagnosed based on the measured activity value. For example, using the synovial fluid collected from the joint of a subject as a sample, RA can be diagnosed and estimated, for example, by incubating synovial fluid (100 μL) with Arg-Arg-MCA (1 μM) in a reaction mixture comprising 50 mM Tris hydrochloric acid (pH 7.4, 1 mL) containing bestatin (30 μg/mL), in the absence or presence of tynorphin at 37° C. for 30 minutes and measuring the fluorescence (excitation wavelength: 360 nm, fluorescence wavelength: 440 nm) of the solution after incubation. In the case where the activity of the dipeptidyl peptidase is not less than about 20 pmol/30 min/mg protein, preferably about 30 to 200 pmol/30 min/mg protein, the patient can be diagnosed and estimated as RA.

Examples of the dipeptidyl peptidase that can be measured by the above-mentioned measurement of the activity of the dipeptidyl peptidase may include, for example, dipeptidyl peptidase III (DPPIII) of enkephalin-degrading enzyme, dipeptidyl peptidase I (DPPI) of lysosome cystein protease, dipeptidyl peptidase II (DPPII), dipeptidyl peptidase IV (DPPIV) and the like, but are not limited thereto. Even if the dipeptidyl peptidase used is any type, RA can be diagnosed and estimated so long as the measured activity of the dipeptidyl peptidase by the above-mentioned method is high.

Furthermore, the present invention also provides a method for the diagnosis of RA, which comprises measuring the amount of spinorphin in the sample in addition to the above-mentioned measurement of the activity of the dipeptidyl peptidase.

The measurement of the amount of spinorphin in the sample comprises the following steps (A) to (E):
(A) mixing a sample collected from a living body with trichloroacetic acid to give a solution phase, and subjecting said solution phase and a solvent to a reverse phase column chromatography to elute spinorphin with said solvent;
(B) contacting the spinorphin eluted in the step (A) with spinorphin immobilized on a carrier and a spinorphin antibody;
(C) removing the spinorphin antibody that has not been bound to the spinorphin immobilized on the carrier;
(D) contacting the spinorphin antibody bound to the spinorphin immobilized on the carrier obtained in the step (C) with a labeled secondary antibody that specifically binds to said spinorphin antibody so as to binding the spinorphin antibody bound to the spinorphin immobilized on the carrier and the labeled secondary antibody; and
(E) measuring the amount of label in the labeled secondary antibody bound to the spinorphin antibody.

In the step (A), a sample collected from a living body is mixed with trichloroacetic acid to give a solution phase, and said solution phase is then subjected to a reverse phase column chromatography to elute spinorphin with a solvent.

Although the usage form of trichloroacetic acid varies depending on the kind of the sample collected from a living body to be measured, it is preferable to use an aqueous solution of trichloroacetic acid comprising trichloroacetic acid of about 5 to 20% by mass. The amount of trichloroacetic acid is preferably an amount sufficient to precipitate proteins in the sample collected from a living body. It is preferable to add the aqueous solution of trichloroacetic acid in an amount of about 0.5 to 2 volume ratio relative to 1 volume of the solution of the sample collected from a living body. The solution phase can be separated by a known method such as filtration, centrifugation or the like. Preferable examples of the column for the reverse phase column chromatography may include an ODS column and the like. The ODS column chromatography is carried out using a column filled with a filler in which octadecylsilyl groups (ODS groups, C18 groups) have been chemically bound to a silica gel carrier. Examples of the ODS column may include ODS-A 60-60/30 (manufactured by YMC Co., Ltd.), Inertsil ODS (manufactured by GL Sciences, Inc.), L-column ODS (manufactured by Chemicals Evaluation and Research Institute), Develosil ODS UG-5 (manufactured by Nomura Chemical Co., Ltd.), CAPCELL PAK C18 MGII (manufactured by Shiseido Co., Ltd.), ZORBAX XDB-C18 (manufactured by Yokogawa Analytical Systems Inc.), Symmetry C18 (manufactured by Waters, Inc.), Nucleosil C18 (manufactured by M. Nagel Co., Inc.) and the like.

Examples of the solvent to be used to elute spinorphin may include alcohols having 1 to 5 carbon atom(s) (e.g., methanol, ethanol, propanol and the like) and acetonitrile, a mixed solvent of two or more kinds of these solvents, and a mixed solvent of the above-mentioned solvent and water.

Secondly, in the step (B), the spinorphin eluted in the step (A) is contacted with the spinorphin immobilized on a carrier and a spinorphin antibody.

Examples of the carrier on which spinorphin is to be immobilized may include a microtiter plate, a test tube, polymer beads and the like. Since spinorphin is an oligopeptide, a carrier having active functional groups such as amino groups, carboxyl groups and the like on the surface, and a carrier having a coat slide of a hydrophobic polymer (e.g., Immuno Plate, MaxiSorp (manufactured by Nalge Nunc International)) and the like are preferred. It is preferable that spinorphin is immobilized on a carrier by covalent bonding of spinorphin to the surface of the carrier. It is preferable that said bonding is carried out by dissolving spinorphin in a buffer such as phosphate buffer, Tris-HCl saline or the like, contacting said solution with the carrier, and incubating the carrier at the temperature of about 0 to 50° C., preferably at room temperature, for at least not less than about 30 minutes, preferably about 30 to 120 minutes. It is preferable that the carrier on which spinorphin has been immobilized is blocked with a blocking agent. Examples of the blocking agent may include skim milk, Block Ace (trade mark) and the like.

Spinorphin can be produced by a known method, for example, the method described in JP-A-2000-95794.

As a preferable spinorphin antibody used for the present invention, any antibody can be used so long as it specifically binds to spinorphin. The spinorphin antibody can be prepared easily by sensitizing an animal (e.g., rabbit, rat or the like) using spinorphin as an antigen, and separating and purifying the obtained antibody by a conventional method. It is preferable to conjugate spinorphin with a carrier protein so as to increase immunogenicity, because spinorphin is a peptide consisting of seven amino acids and is generally too short to exhibit immunogenicity. Examples of such carrier protein for conjugation may include, for example, KLH (Keyhole Limpet Hemocyanin), bovine serum albumin, ovalbumin and the like. Examples of the method for conjugation may include a method comprising activating the C-terminal carboxyl group of spinorphin with a carbodiimide (e.g. 1-ethyl-3-(3-dimethylaminopropyl)carbodiimide, etc.) and reacting the activated spinorphin with a primary amine of a carrier protein (EDC method), a method comprising binding an amino group of a protein and an SH group of a peptide (spinorphin) using MBS (maleimide benzoyloxysuccinimide)-based crosslinking agent (MBS method) and the like.

As the antibody prepared by the above-mentioned method, either a polyclonal antibody or a monoclonal antibody can be used.

It is preferable that the spinorphin antibody obtained as above is specific to spinorphin. It is preferable that the spinorphin antibody does not cross-react with spinorphin analogues such as VVYPWT (sequence listing: SEQ ID NO: 2), VYPWT (sequence listing: SEQ ID NO: 3), hemorphin-4 (YPWT; sequence listing: SEQ ID NO: 4), PWT, LVVYPW (sequence listing: SEQ ID NO: 5), tynorphin (VVYPW; sequence listing: SEQ ID NO: 6), VYPW (sequence listing: SEQ ID NO: 7), YPW, LVVYP (sequence listing: SEQ ID NO: 8), VVYP (sequence listing: SEQ ID NO: 9), VYP and the like.

In the step (B), it is preferable that the contact of spinorphin eluted in the step (A) with the spinorphin immobilized on the carrier and the spinorphin antibody is performed by contacting a solution wherein the above-mentioned spinorphin rabbit antibody and the spinorphin eluted in the step (A) have been dissolved or optionally diluted in a buffer (e.g., Tris buffer saline, phosphate buffer and the like), with a carrier on which spinorphin has been immobilized. Such contact is preferably carried out at usually 0° C. to room temperature, while allowing to stand or shaking slowly, for about 30 minutes to 2 hours, preferably about 30 minutes to 1 hour. During said reaction, the spinorphin immobilized on a carrier and the spinorphin eluted in the step (A) compete with the spinorphin antibody so that the spinorphin immobilized on a carrier can bind to the spinorphin antibody.

Secondly, in the step (C), the spinorphin antibody that has not been bound to the spinorphin immobilized on a carrier is removed.

It is preferable that said removal is carried out, for example, by decanting the carrier to discard the solution contacting with the carrier, or aspirating the solution using a pipette. It is preferable that the carrier is further washed with the above-mentioned buffer and the like at least 3 times, preferably about 3 to 6 times.

Then, in the step (D), the spinorphin antibody bound to the spinorphin immobilized on the carrier obtained in the step (C) is contacted with a labeled secondary antibody that specifically binds to said spinorphin antibody so as to binding the spinorphin antibody bound to the spinorphin immobilized on the carrier and the labeled secondary antibody.

Examples of the labeled secondary antibody may include anti-rabbit IgG goat IgG antibody, goat anti-mouse antibody and the like that are labeled with a label enzyme or a fluorescence dye. Examples of the label enzyme may include peroxidase, glucose oxidase, acidic phosphatase, alkaline phosphatase and the like, preferably peroxidase, and especially preferably horseradish peroxidase. Examples of the labeling fluorescence dye may include MFP 488, AlexaFluor 488, rhodamine, fluoresceine, Cy2, Cy3 and the like.

The binding between the spinorphin antibody bound to the spinorphin immobilized on a carrier and the labeled secondary antibody is carried out usually at 0° C. to room temperature, while allowing to stand or shaking slowly, for about 30 minutes to 2 hours, preferably about 30 minutes to 1 hour.

In the step (E), the amount of label in the labeled secondary antibody bound to the spinorphin antibody in the step (D) is measured.

In the case where a secondary antibody labeled with an enzyme, for example, is used, the amount of label in the labeled secondary antibody bound to the spinorphin antibody bound to the immobilized antigen (spinorphin) can be measured by measuring the light absorbance at the wavelength corresponding to the color tone of the color reaction between the label enzyme and the substrate. The color reaction between the label enzyme and the substrate can be carried out by contacting the substrate solution with the enzyme-labeled secondary antibody. The substrate differs depending on the label enzyme to be used. In the case where the label enzyme is, for example, horseradish peroxidase, examples of the preferable substrate to be used may include o-phenylenediamine, 3,3′,5,5′-tetramethylbenzidine and the like. It is preferable that the light absorbance is measured within about 10 minutes to 60 minutes after coloring.

In the case where fluorescence labeling is used, the fluorescence intensity may be measured using a fluorometer.

In the case where the carrier is a microplate, it is preferable to use an autoreader or a microplate reader that can measure light absorbance successively and automatically in the case where enzyme labeling is used, and it is preferable to use a fluorescence microreader or the like in the case where fluorescence labeling is used.

In the above-mentioned method, spinorphin can be measured quantitatively in the concentration range of preferably about 10⁻¹⁰ to 10⁻⁷ g/mL. Accordingly, in the case where a sample comprising spinorphin at high concentration is used, it is preferable to dilute the sample as appropriate with the above-mentioned buffer and the like.

By using the method for the measurement of the present invention, for example, in the case where the amount of spinorphin in the synovial fluid collected from the joint of a subject is measured to be about not more than 5 ng/mL, the subject can be estimated to be suffering from rheumatoid arthritis.

Subsequently, the above-mentioned activity of the dipeptidyl peptidase and the amount of spinorphin can be analyzed statistically as follows. For example, the activity of the dipeptidyl peptidase and the concentration of spinorphin in the synovial fluid from an RA patient and an OA patient can be compared by, for example, Mann Whitney U test. As a result of comparison, in the case where the risk rate is not more than 5%, there may be a significant difference. According to the present invention, a negative correlation can be obtained by, for example, plotting the activity of the dipeptidyl peptidase at the vertical axis and the amount of spinorphin at the horizontal axis. From this fact, RA can be diagnosed and estimated with high probability by diagnosing and estimating from the characteristic values of both the activity of the dipeptidyl peptidase and the amount of spinorphin. Specifically, for example, in the case where the synovial fluid in the joint of the subject is used as a sample and the activity of the dipeptidyl peptidase in the synovial fluid is not less than about 20pmol/30 min/mg protein, preferably about 30 to 200 pmol/30 min/mg protein, and the amount of spinorphin is not more than 5 ng/mL, the probability of RA is very high.

In the kit for diagnosis of RA of the present invention, examples of the fluorescence substrate that is specific to the dipeptidyl peptidase may include, for example, synthetic fluorescence substrates having an Arg-Arg group such as Arg-Arg-4-methylcoumaryl-7-amide, Boc-Gly-Arg-Arg-4-MCA and the like.

Examples of the aminopeptidase inhibitor may include bestatin, leuhistin and the like.

Examples of the dipeptidyl peptidase inhibitor may include tynorphin and the like.

According to the kit for the diagnosis of RA of the present invention, whether the person who provides a sample is suffering from RA or not can be diagnosed readily and accurately within a short period of time.

In the present invention, besides the above-mentioned kit for diagnosis of RA, a kit comprising a spinorphin antibody, an antigen for coating (spinorphin), a microtiter plate, a labeled secondary antibody and other reagents may also be used for the measurement of the concentration of spinorphin in a sample.

For the kit for the measurement of the concentration of spinorphin, examples of the spinorphin antibody may include a spinorphin rabbit antibody and the like. Examples of the antigen for coating may include a solution of spinorphin in a buffer. Preferable examples of the microtiter plate may include a microtiter plate having active functional groups such as amino groups, carboxyl groups and the like on the surface, a microtiter plate having a coat slide of a hydrophobic polymer. Alternatively, a kit comprising a microtiter plate on which spinorphin as an antigen has been immobilized, a spinorphin antibody and a labeled secondary antibody instead of an antigen for coating and a microtiter plate may be used. Examples of the preferable labeled secondary antibody may include the above-mentioned enzyme-labeled secondary antibody and fluorescence-labeled secondary antibody. Further, in the case where an enzyme-labeled secondary antibody is included in the kit, other reagents may include, for example, the above-mentioned substrate and the like, a washing liquid for plate (such as a buffer added with a surfactant (e.g., Polysorbate 20, etc.), etc.), a buffer (e.g., phosphate buffer, Tris buffer saline) and the like.

According to the above-mentioned kit for the measurement of the concentration of spinorphin, the concentration of spinorphin in the sample can be measured readily and accurately within a short period of time.

### EXAMPLES

Hereinafter, the present invention is explained by way of Examples, but the present invention is not limited thereto. Reagent:

Spinorphin and tynorphin were obtained from the American Peptide Company Inc., Sunnyvale, Calif., USA.

Arg-Arg-MCA and bestatin were purchased from Sigma-Aldrich Fine Chemical Co., St. Louis, Mo., USA. HRP-F (ab′)₂ goat anti-rabbit IgG (H+L) was purchased from Zymed Laboratories, Inc., Carlton Court, Calif., USA. As the ODS column, ODS-A 60-60/30 (manufactured by YMC Co., Ltd., Kyoto, Japan) was used. All of other reagents used were of high grade.

Collection of Samples:

Synovial fluid was collected from OA patients (40 persons) and RA patients (39 persons). A sample of the synovial fluid was sucked from the knee joint of an outpatient, placed into a plastic container and centrifuged at 4° C. for 10 minutes at 1500×g. The supernatant was kept at −20° C. until analysis. Diagnosis of OA and RA was based on the “ACR Revised Diagnosis Criteria” by the American College of Rheumatology (ACR).

The ages of OA patients were 74 to 88 years old (11 men and 29 women), and their average morbid period was 6 years (range: 0.1 to 20 years). The ages of RA patients were 44 to 74 years old (6 men, 32 women), and their average morbid period was 14.5 years (range: 1 to 36 years, least erosive subset (LES) 3 persons, more erosive subset (MES) 29 persons, multilating disease (MUD) 6 persons). All of the RA patients took several kinds of medicines. The medicines were an anti-inflammatory agent, a gold compound, methotrexate, sulfasalazine, a corticosteroid, bucillamine and D-penicillamine. There was no person who had received a corticosteroid or an intra-articular steroid at high concentration before sucking of the synovial fluid. The approval from each patient was reviewed by the Committee, and individual informed concept was obtained from each patient.

The amount of protein in the synovial fluid was measured by the Bradford method (Pierce Inc., Rockford, USA). Statistical processing:

The activity of the dipeptidyl peptidase and the concentration of spinorphin in the synovial fluid of RA patients and OA patients were determined by the Mann Whitney U test, and significant difference was recognized where the risk rate is not more than 5%.

### EXAMPLES

### Example 1

Dipeptidyl Peptidase Activity:

Dipeptidyl peptidase activity was measured by hydrolysis of Arg-Arg-MCA on a specific synthetic fluorescence substrate. Firstly, 50 mM Tris-HCl buffer comprising bestatin (30 μg/mL) and Arg-Arg-MCA (1 μM) (pH 7.4, 1 mL) was pre-incubated at 37° C. for 30 minutes. After the pre-incubation was completed, the synovial fluid (100 μL) was added to the buffer and mixed, and the mixture was incubated in the presence or absence of a dipeptidyl peptidase inhibitor (100 μg/mL) at 37° C. for 30 minutes. The reaction was stopped by adding 50% (v/v) aqueous acetic acid. The mixture was allowed to stand at 4° C. for 10 minutes, and the fluorescence intensity of the liberated AMC (which shows the dipeptidyl peptidase activity) was measured using a fluorescence spectrum spectrometer F-2000 (manufactured by Hitachi Co. Ltd, Tokyo, Japan) at the excitation wavelength of 360 nm and the fluorescence wavelength of 440 nm.

The dipeptidyl peptidase activity was calculated by the following equation:
Activity=(Activity of substrate degradation in the absence of a dipeptidyl peptidase inhibitor)−(Activity of substrate degradation in the presence of a dipeptidyl peptidase inhibitor).

As the dipeptidyl peptidase inhibitor, tynorphin was used.

The above-mentioned activity of substrate degradation was converted to a value per 1 mg of protein in the sample by depicting a standard curve based on the relationship between the concentration of AMC and the value of fluorescence, and calculating the value from the standard curve.

Results:

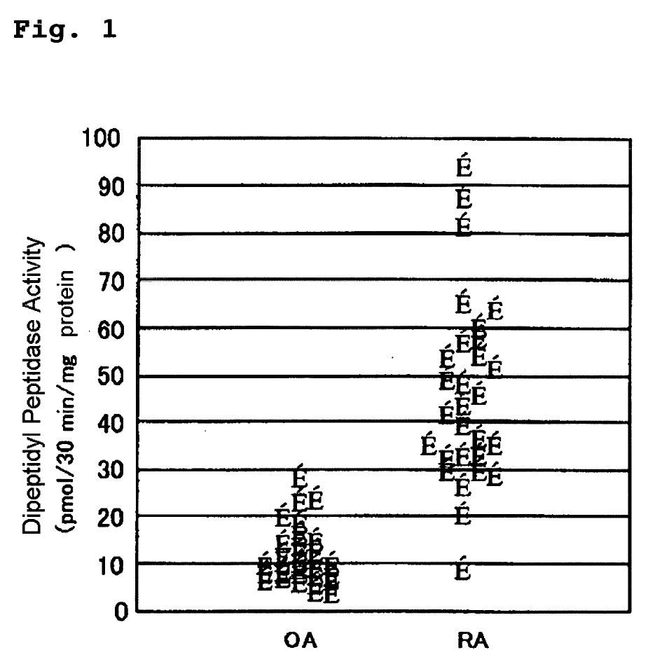

The dipeptidyl peptidase activities in the synovial fluid from RA patients and OA patients were 46.1±19.6 pmol/30 min/mg protein (n=29) and 10.6±6.2 pmol/30 min/mg protein (n=33), respectively (FIG. 1).

The dipeptidyl peptidase activity of the RA patients was about 5-fold higher than that of the OA patients (p<0.001).

There was no correlation between the dipeptidyl peptidase activity of RA and severity of the disease, the morbid period or the values in the serum test such as C-reaction type protein and blood sedimentation ratio and the like.

### Example 2

Measurement of Spinorphin

Synovial fluid sample (2 mL) was mixed with the equivalent amount of trichloroacetic acid (10% by mass), and the mixture was allowed to stand at 4° C. for 1 hour.

The mixture was centrifuged at 1500×g for 20 minutes, and the supernatant was subjected to an ODS column. The column was washed with water (10 volume-fold), and spinorphin was eluted with 80% (v/v) aqueous methanol solution. The solvent was evaporated, and the eluate was dissolved in Tris buffer saline (TBS, 0.5 mL) to prepare a sample.

Spinorphin (50 ng) and TBS (100 μL) were placed in a 96-well plate (Nunc-Immuno Plate, MaxiSorp Surface, Nalge Nunc International, Denmark; hereinafter also referred to simply as a plate) and reacted at room temperature for 1 hour to immobilize spinorphin on the plate. The plate on which spinorphin had been immobilized was washed five times with 10 mM TBS (TBS-T) comprising 0.1% by mass of Tween 20. The plate on which spinorphin had been immobilized was then blocked with TBS-T comprising skim milk (10% by mass) and sodium azide (0.1% by mass) at room temperature for 1 hour. Skim milk was removed from the plate by washing. A spinorphin rabbit antibody (5 μg/well) was added to the plate, a diluted solution of the sample (100 μL) was added thereto, and the mixture was subjected to competitive reaction. The plate was reacted for 1 hour under shaking slowly. The plate was then washed five times. Horseradish peroxidase (HRP)-labeled F (ab′)₂ goat anti-rabbit IgG (H+L) (1000-fold dilution, 100 μL) was added to the plate and reacted at room temperature for 1 hour. After the reaction was completed, the plate was washed five times, and o-phenylenediamine (2.2 μM) and citric acid buffer comprising 0.014% (v/v) aqueous hydrogen peroxide (0.1M, pH 5, 100 μL) were then added to the plate. The plate was allowed to stand for 20 minutes, and the light absorbance of the solution in the wells of the plate using an autoreader (Korona Electric Co. Ltd., Ibaragi, Japan) at 405 nm. The concentration of spinorphin in the sample was calculated from % B/Bo of the standard curve (light absorbance ratio; a value obtained by dividing the light absorbance with the light absorbance at blank 0).

Results:

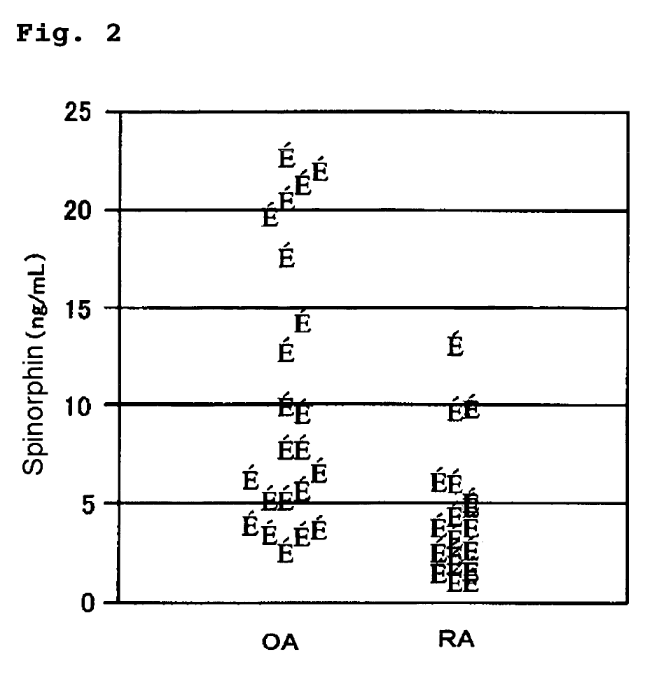

The amount of spinorphin in the synovial fluid from the RA patients was 4.2±3.4 ng/mL (n=20) and that of the OA patients was 10.1±7.1 ng/mL (n=23), respectively (FIG. 2). The amount of spinorphin in the RA patients was significantly lower (P<0.01) than that in the OA patients.

In the 20 samples, the dipeptidyl peptidase activity and the amount of spinorphin in the synovial fluid had a negative correlation (r=−0.51) where both the dipeptidyl peptidase activity and the amount of spinorphin could be measured.

### Reference Example

Production of a Spinorphin Rabbit Antibody

Spinorphin (obtained from American Peptide Company Inc., Sunnyvale, Calif., USA) was bound to KLH (keyhole limpet hemocyanin) using EDC (1-ethyl-3-(3-dimethylaminopropyl)carbodiimide) to give a KLH-bound spinorphin. The KLH-bound spinorphin was injected subcutaneously to the backs of two rabbits (New Zealand white). The KLH-bound spinorphin was immunized 8 times at the intervals of 2 weeks. After the last immunization was completed, blood was collected from the heart of the rabbit. Serum was separated from the collected whole blood to give a spinorphin rabbit antibody. The titer of the antibody was measured by reacting a solid antigen (200 ng/well) with diluted serum (100 μL/well), adding the secondary antibody after washing, finally adding the substrate, and measuring the coloring. The serum was diluted firstly 1000-fold, then every 3-fold and at the maximum 81000-fold to prepare a diluted serum, and said diluted serum was used for the reaction.

The cross reaction of anti-spinorphin exhibited by spinorphin analogues [VVYPWT (sequence listing: SEQ ID NO: 2), VYPWT (sequence listing: SEQ ID NO: 3), hemorphin-4 (YPWT; sequence listing: SEQ ID NO: 4), PWT, LVVYPW (sequence listing: SEQ ID NO: 5), tynorphin (VVYPW; sequence listing: SEQ ID NO: 6), VYPW (sequence listing: SEQ ID NO: 7), YPW, LVVYP (sequence listing: SEQ ID NO: 8), VVYP (sequence listing: SEQ ID NO: 9) and VYP] was lower than 0.1% of that of cross reaction of spinorphin. Therefore, a cross reaction was not recognized. The spinorphin analogues were synthesized according to the method disclosed in JP-A-2000-95794.

### INDUSTRIAL APPLICABILITY

The present invention is useful as a novel method for the diagnosis of RA.

1. A method for the diagnosis of rheumatoid arthritis, comprising measuring the activity of a dipeptidyl peptidase in a sample collected from a mammal.

2. The method for the diagnosis as claimed in claim 1, wherein the sample is at least one member selected from joint synovial fluid, blood and cerebrospinal fluid.

3. The method for the diagnosis as claimed in claim 1, further comprising measuring an amount of spinorphin in the sample collected from a mammal.

4. The method for the diagnosis as claimed in claim 3, wherein rheumatoid arthritis disease is estimated at a spinorphin concentration of not more than 5 ng/mL in the measured sample.

5. A method for measuring the activity of a dipeptidyl peptidase for the diagnosis as claimed in claim 1, comprising reacting a fluorescence substrate specific to a dipeptidyl peptidase and an aminopeptidase inhibitor with a sample in the presence or absence of a dipeptidyl peptidase inhibitor, and measuring the fluorescence intensity of the reaction solution after the reaction.

6. The measuring method as claimed in claim 5, wherein the fluorescence substrate is a synthetic fluorescence substrate having an Arg-Arg group.

7. The measuring method as claimed in claim 6, wherein the synthetic fluorescence substrate is Arg-Arg-4-methylcoumaryl-7-amide.

8. The measuring method as claimed in claim 7, comprising measuring the fluorescence intensity of 7-amino-4-methylcoumarin produced by the reaction.

9. The measuring method as claimed in claim 5, wherein the aminopeptidase inhibitor is bestatin.

10. The measuring method as claimed in claim 5, wherein the dipeptidyl peptidase inhibitor is tynorphin.

11. A kit for the diagnosis of rheumatoid arthritis, comprising a fluorescence substrate specific to a dipeptidyl peptidase, an aminopeptidase inhibitor and a dipeptidyl peptidase inhibitor.

12. The kit as claimed in claim 11, wherein the fluorescence substrate is a synthetic fluorescence substrate having an Arg-Arg group.

13. The kit as claimed in claim 12, wherein the synthetic fluorescence substrate is Arg-Arg-4-methylcoumaryl-7-amide.

14. The method for the diagnosis as claimed in claim 2, further comprising measuring an amount of spinorphin in the sample collected from a mammal.

15. The method for the diagnosis as claimed in claim 14, wherein rheumatoid arthritis disease is estimated at a spinorphin concentration of not more than 5 ng/mL in the measured sample.

16. The measuring method as claimed in claim 6, wherein the aminopeptidase inhibitor is bestatin.

17. The measuring method as claimed in claim 7, wherein the aminopeptidase inhibitor is bestatin.

18. The measuring method as claimed in claim 6, wherein the dipeptidyl peptidase inhibitor is tynorphin.

19. The measuring method as claimed in claim 7, wherein the dipeptidyl peptidase inhibitor is tynorphin.

20. The measuring method as claimed in claim 8, wherein the dipeptidyl peptidase inhibitor is tynorphin.


SEQUENCE LISTING (9 sequences)

SEQ ID NO: 1 - protein, 7 aa, Artificial Sequence
  Descripton of Artificial Sequence Synthetic Peptide
LVVYPWT

SEQ ID NO: 2 - protein, 6 aa, Artificial Sequence
  Descripton of Artificial Sequence Synthetic Peptide
VVYPWT

SEQ ID NO: 3 - protein, 5 aa, Artificial Sequence
  Descripton of Artificial Sequence Synthetic Peptide
VYPWT

SEQ ID NO: 4 - protein, 4 aa, Artificial Sequence
  Descripton of Artificial Sequence Synthetic Peptide
YPWT

SEQ ID NO: 5 - protein, 6 aa, Artificial Sequence
  Descripton of Artificial Sequence Synthetic Peptide
LVVYPW

SEQ ID NO: 6 - protein, 5 aa, Artificial Sequence
  Descripton of Artificial Sequence Synthetic Peptide
VVYPW

SEQ ID NO: 7 - protein, 4 aa, Artificial Sequence
  Descripton of Artificial Sequence Synthetic Peptide
VYPW

SEQ ID NO: 8 - protein, 5 aa, Artificial Sequence
  Descripton of Artificial Sequence Synthetic Peptide
LVVYP

SEQ ID NO: 9 - protein, 4 aa, Artificial Sequence
  Descripton of Artificial Sequence Synthetic Peptide
VVYP